(19) United States
(12) Patent Application Publication
(10) Pub. No.: US 2007/0299054 A1
(43) Pub. Date: Dec. 27, 2007

(54) Oral pharmaceutical composition of a poorly water-soluble active agent

(76) Inventors: Rajesh Jain, New Delhi (IN); Kour C. Jindal, New Delhi (IN); Amarjit Singh, New Delhi (IN); Munish Talwar, New Delhi (IN); Henricus R.M. Gorissen, Weesp (NL)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/812,763

(22) Filed: Jun. 21, 2007

Related U.S. Application Data
(60) Provisional application No. 60/815,589, filed on Jun. 22, 2006.

(51) Int. Cl. A61K 31/55; A61K 31/675

(52) U.S. Cl. 514/212.07; 514/81

(57) ABSTRACT

The present invention relates to an improved oral pharmaceutical composition containing at least one poorly water soluble active agent, the active agent containing at least one of an endothelin conversion enzyme (ECE) inhibitor and a neutral endopeptidase (NEP) inhibitor in an amount greater than 10% w/w of the composition, and an alkali system comprising a mixture of at least two alkaline compounds in a ratio of from 1:20 to 20:1. The present invention also relates to an improved oral pharmaceutical composition containing, SLV-306 or at least one pharmaceutically acceptable salt, ester, hydrate, solvate, isomer or derivative thereof, as an active agent, and an alkali system in an amount greater than 10% w/w of the composition comprising a mixture of at least two alkaline compounds and optionally at least one pharmaceutically acceptable excipient. The present invention further relates to a process for preparation of such improved compositions and methods for treatment using such compositions.

This application claims the benefit of priority of U.S. Provisional Application No. 60/815,589, filed on Jun. 22, 2006, the disclosure of which is incorporated by reference herein.

The present invention relates to an improved oral pharmaceutical composition comprising at least one poorly water soluble active substance (also referred to as an active agent) in an amount greater than 10% w/w of the composition, wherein the active agent comprises at least one agent chosen from an endothelin conversion enzyme (ECE) inhibitor and a neutral endopeptidase (NEP) inhibitor, and an alkali system in an amount greater than 10% w/w of the composition, wherein the alkali system comprises a mixture of at least two alkaline compounds and optionally at least one pharmaceutically acceptable excipient.

In one embodiment of the present invention, the oral pharmaceutical composition comprises an alkali system comprising a mixture of at least two alkaline compounds in a ratio of from 1:20 to 20:1, and an the active agent of formula (I):

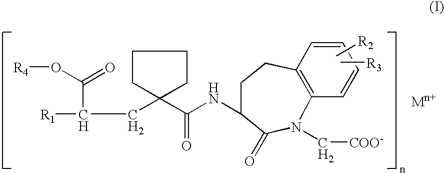

or at least one pharmaceutically acceptable hydrate or solvate or a mixture thereof,

wherein:
R₁ is a (C₁-C₆)alkoxy(C₁-C₆)alkyl group, which is optionally substituted with at least one of a (C₁-C₆)alkoxy group, a phenyl-(C₁-C₆)-alkyl group or a phenyloxy-(C₁-C₆)-alkyl group, wherein the phenyl group may be substituted with at least one of a (C₁-C₆)alkyl group, a (C₁-C₆)alkoxy group, a halogen atom, or a naphtyl-(C₁-C₆)-alkyl group;
R₂ and R₃, which are the same or different, are chosen from a hydrogen atom and a halogen atom;
R₄ is a biolabile ester forming group;
M is chosen from a hydrogen atom and a metal ion, such as a bivalent metal ion; and
n is chosen from 1, 2 and 3.

In another embodiment of the invention, the oral pharmaceutical composition comprises an alkali system comprising a mixture of at least two alkaline compounds in a ratio of from 1:20 to 20:1, and an active agent, wherein the active agent is 1H-1-Benzazepine-1-acetic acid, 3-[[[1-[2-(ethoxycarbonyl)-4-phenylbutyl]cyclopentyl]carbonyl]amino]-2,3,4,5-tetrahydro-2-oxo- (SLV 306). In another embodiment of the invention, the oral pharmaceutical composition comprises, as an active agent, SLV-306 in its 3S,2′R form. In yet another embodiment of the invention, the oral pharmaceutical composition comprises, as an active substance, SLV-306 as its Ca2+ salt, or for example, as its pharmaceutically acceptable hydrate or solvate.

In the present invention, R₄ in formula (I), which is a biolabile ester forming group, is, for example, chosen from lower alkyl groups, phenyl or phenyl-lower-alkyl groups, which are optionally substituted in the phenyl ring by lower alkyl or by a lower alkylene chain bonded to two adjacent carbon atoms, dioxolanylmethyl groups, which are optionally substituted in the dioxolane ring by lower alkyl groups, and C2-C6-alkanoyloxymethyl groups, which are optionally substituted on the oxymethyl group by lower alkyl groups. In some embodiments of the present invention, R₄ in formula (I), which is a biolabile ester forming group, is a lower alkyl group, and the lower alkyl group is, for example, an unbranched alkyl group with 1 to 4, for example 2, carbon atoms. In some embodiments of the present invention, R₄ in formula (I), which is a biolabile ester group, is an optionally substituted phenyl-lower-alkyl group, wherein its alkylene chain may contain 1 to 3, for example 1, carbon atoms. In other embodiments of the present invention, R₄ in formula (I), which is a biolabile ester forming group, is a phenyl or phenyl-lower-alkyl group, wherein the phenyl ring is substituted by a lower alkylene chain, wherein the alkylene chain may contain 3 to 4, for example 3, carbon atoms. In embodiments of the present invention in which R₄ in formula (I), which is a biolabile ester forming group, is a phenyl-containing substituent, the phenyl-containing substituent is chosen from phenyl, benzyl and indanyl. In other embodiments in which R₄ in formula (I), which is a biolabile ester forming group, is an optionally substituted alkanoyloxymethyl group, the alkanoyloxy group may contain, for example 2 to 6, such as 3 to 5, carbon atoms and may be branched, and can be, for example, a pivaloyloxymethyl radical (tert-butylcarbonyloxymethyl radical).

The compositions of the present invention are easier to formulate and possess improved solubility and stability. The present invention also describes a process for preparation of such improved compositions and a method for treatment using such compositions.

The accompanying drawings, which are incorporated in and constitute a part of this specification, illustrate several embodiments of the invention and, together with the description, serve to explain the principles of the invention.

### BRIEF DESCRIPTION OF THE DRAWING

FIG. 1 is a graph comparing release profiles for tablets of various formulations.

Endothelins (ETs) are potent vasoconstrictors, promitogens, and inflammatory mediators. They have been implicated in the pathogenesis of various cardiovascular, renal, pulmonary, and central nervous system diseases. Since the final step of the biosynthesis of ETs is catalyzed by a family of endothelin-converting enzymes (ECEs), inhibitors of these enzymes may represent novel therapeutic agents. Currently, seven isoforms of these metalloproteases have been identified; they all share an amino acid sequence identity with neutral endopeptidase (NEP), another metalloprotease. Therefore, the majority of ECE inhibitors also possess potent NEP inhibitory activity. To date, three classes of ECE inhibitors have been synthesized: dual ECE/NEP inhibitors, triple ECE/NEP/ACE inhibitors, and selective ECE inhibitors. An agent which suppresses endothelin production, such as an ECE inhibitor, or which inhibits the binding of endothelin to an endothelin receptor, such as an endothelin receptor antagonist, antagonizes various physiological effects of endothelin and produces beneficial effects in a variety of therapeutic areas. Endothelin receptor antagonists and ECE inhibitors are therefore useful in treating a variety of diseases affected by endothelin. Some examples of these diseases include, but are not limited to, chronic heart failure, myocardial infarction, cardiogenic shock, systemic and pulmonary hypertension, ischemia-repurfusion injury, atherosclerosis, coronary and systemic vasospastic disorders, cerebral vasospasm, and subarachnoid hemorrhage and the like.

SLV-306 (daglutril) is an orally active inhibitor of neutral endopeptidase (NEP) and endothelin conversion enzyme (ECE). It belongs to the class of benzazepine, benzoxazepine and benzothiazepine-N-acetic acid derivatives which contain an oxo group in the alpha position relative to the nitrogen atom and are substituted in position 3 by a 1-(carboxyalkyl) cyclopentyl-carbonylamino radical. These compounds and their salts and biolabile esters are within the scope of the present invention and have NEP-inhibitory effects on the heart, as described in Waldeck et al., U.S. Pat. No. 5,677,297 and EP 0733642. The benzazepine-N-acetic acid compounds used in the present invention are known from EP 0733642, EP 0830863, WO 00/48601 and WO 01/03699, and can be produced by the methods described in U.S. Pat. No. 5,677,297 and EP 0733642. These patents are related to these compounds and their physiologically acceptable salts as such and to the use of the compound in heart insufficiency. WO 03/059939 relates to specific salts of these compounds, especially to the calcium salt. EP 0830863, WO00/48601 and WO01/03699 are related to the use of the above compounds in the improvement of gastrointestinal blood flow, in the treatment of hypertension and in the treatment and prophylaxis of cardiac damages induced by adriamycin and comparable anti-cancer drugs, respectively.

Various active substances have a very poor solubility in gastric fluid. When these active substances are administered to the body, they often have a poor bio-availability due to the poor solubility in the digestive fluid. In order to solve this problem several methods were developed, such as micronization, inclusion in cyclodextrins, the use of inert water-soluble carriers, the use of solid dispersions (WO 00/00179) or solid solutions or nanocrystalline or amorphous forms of an active substance. Also the compounds described in U.S. Pat. No. 5,677,297 and EP 0733642, including SLV-306, are drugs with poor bioavailability due to their poor solubility in gastric fluid. Even when SLV-306 is used in its salt form, it forms a gel like structure in the acid gastric fluid. The gel like structure formed is very difficult to solubilize again even under alkaline conditions, leading to a low overall bioavailability.

WO 03/068266 describes an oral solid solution formulation of compounds of formula (I) having enhanced bio-availability compared with said active substance in a traditionally formulated form. Although this formulation has superior bioavailability properties, it has the draw-back that it is formed via a melt mixture leading to some restrictions; it has to be formulated either into a capsule, or into a tablet via melt-extrusion technique. Further the size of the formulation will be too large for higher dosages.

WO 06/067150 (not pre-published) describes an oral immediate release formulation of compounds of formula (I) comprising the active substance in an amount up to 60% of the total weight of the formulation, at least 10% w/w of an alkaline compound or a mixture of alkaline compounds, between 0.1 and 10% w/w of one or more surfactants and optionally auxiliary materials in an amount of from 1% to 45% of the total weight of the formulation. Especially when docusate sodium is used as the surfactant a good bioavailability of the active substance is obtained.

One objective of the present invention is to provide a new oral formulation for compounds with a low oral bioavailability, for example, compounds containing one or more endothelin conversion enzyme (ECE) inhibitors and/or neutral endopeptidase (NEP) inhibitors, wherein the new oral formulation has a significant increase in bio-availability, as compared with an active substance in the traditionally formulated form. This new oral formulation is sufficiently stable for commercial use and also is useful in the preparation of formulations with a high content of active substance, with a reasonable size, and optionally without the use of a surfactant. It is a further objective of the present invention to provide a formulation which can be prepared using normal formulation procedures and equipment, so that substantial investment is not necessary.

It is another objective of the present invention to provide a process for the preparation of such improved compositions.

It is also an objective of the present invention to provide an improved oral pharmaceutical composition comprising at least one poorly soluble active agent in an amount greater than 10% w/w of the composition, the active agent comprising at least one agent chosen from an endothelin conversion enzyme (ECE) inhibitor and a neutral endopeptidase (NEP) inhibitor other than a compound of the above formula (I), an alkali system in an amount greater than 20% w/w of the composition, and optionally at least one pharmaceutically acceptable excipient.

It is a further objective of the present invention to provide an improved oral pharmaceutical composition comprising at least one poorly soluble active agent in an amount greater than 10% w/w of the composition, the active agent comprising at least one of an endothelin conversion enzyme (ECE) inhibitor and a neutral endopeptidase (NEP) inhibitor, an alkali system in an amount greater than 20% w/w of the composition, the alkali system comprising a mixture of at least two alkaline compounds, and optionally at least one pharmaceutically acceptable excipient.

It is an even further objective of the present invention to provide an improved oral pharmaceutical composition comprising at least one poorly soluble active agent in an amount greater than 10% w/w of the composition, the active agent comprising at least one of an endothelin conversion enzyme (ECE) inhibitor and a neutral endopeptidase (NEP) inhibitor, such as a compound of the above formula (I), an alkali system in an amount greater than 20% w/w of the composition, the alkali system comprising a mixture of at least two alkaline compounds in a ratio of from 1:20 to 20:1, and optionally at least one pharmaceutically acceptable excipient.

It is a further objective of the present invention to provide an improved oral pharmaceutical composition comprising, as an active agent, SLV-306 or at least one pharmaceutically acceptable salt, ester, hydrate, solvate, isomer or derivative thereof, in an amount greater than 10% w/w of the composition, an alkali system in an amount greater than 20% w/w of the composition, the alkali system comprising a mixture of at least two alkaline compounds in a ratio of from 1:20 to 20:1, and optionally at least one pharmaceutically acceptable excipient.

It is another objective of the present invention to provide a process for the preparation of such improved compositions which comprises the following:
i) mixing the active agent and alkali system optionally with at least one pharmaceutically acceptable excipient, and
ii) formulating the mixture produced in (i) into a suitable dosage form.

It is yet another objective of the present invention to provide a method for treating at least one disease chosen from chronic heart failure, myocardial infarction, cardiogenic shock, systemic and pulmonary hypertension, ischemia-repurfusion injury, atherosclerosis, coronary and systemic vasospastic disorders, cerebral vasospasm, and subarachnoid hemorrhage, the method comprising administering an effective amount of the composition to a patient in need thereof.

The improved compositions of the present invention are easier to formulate and possess improved solubility and stability.

In one embodiment, the present invention provides an improved oral pharmaceutical composition comprising at least one poorly soluble active agent, in acid, the at least one active agent, other than a compound of formula (I), is chosen from an endothelin conversion enzyme (ECE) inhibitor and a neutral endopeptidase (NEP) inhibitor, in an amount greater than 10% w/w of the composition, an alkali system in an amount greater than 10% w/w of the composition and optionally at least one pharmaceutically acceptable excipient. In some embodiments of the present invention, the alkaline system comprises a mixture of at least two alkaline compounds.

In the framework of the present invention, surfactants are defined as molecules with well defined polar and non-polar regions that allow them to aggregate in solutions to form micelles. Depending on the nature of the polar area, surfactants can be, for example, non-ionic, anionic, cationic or zwitterionic. Some examples of non-ionic hydrophilic surfactants are polyoxyethylene sorbitan esters, cremophores and poloxamers. Some examples of anionic surfactants are sodium lauryl sarcosinate, docusate and pharmaceutically acceptable docusate salts such as docusate calcium, docusate sodium and docusate potassium.

Inhibitors of neutral endopeptidase (NEP) and/or endothelin conversion enzyme (ECE) within the scope of this invention, include, but are not limited to, CGS 26303, phosphoramidon, FR901533, TMC-66, SM-19712, SLV-306, KC-12615, KC-90095-1-AC, CGS-26303, CGS-30440, CGS-31447, CGS-26670, and Sch-54470, and the pharmaceutically acceptable salts, esters, isomers, derivatives and prodrugs thereof.

In a further embodiment of the present invention, the alkali system comprises an alkaline compound or a mixture of at least two alkaline compounds chosen from, but not limited to, sodium bicarbonate, sodium carbonate, potassium bicarbonate, potassium carbonate, magnesium carbonate, calcium carbonate, tris buffer, triethanolamine; alkaline hydroxides, such as sodium hydroxide, potassium hydroxide or magnesium hydroxide; alkaline phosphates, such as disodium hydrogen phosphate, dipotassium hydrogen phosphate, dicalcium phosphate; and meglumine and mixtures thereof.

In some embodiments of the present invention, the alkali system is present in an amount greater than 10% w/w of the composition, such as an amount greater than 20% w/w, or an amount greater than 30% w/w, 40% w/w, 50% w/w, 55% w/w or 60% w/w of the composition.

In another embodiment of the present invention, the alkali system of the composition comprises a mixture of at least two alkaline compounds in a ratio of from 1:20 to 20:1 w/w.

In another embodiment, the pharmaceutical composition comprises an alkali system comprising a mixture of at least two alkaline compounds in a ratio of from 1:20 to 20:1 w/w and at least one endothelin conversion enzyme (ECE) inhibitor or neutral endopeptidase (NEP) inhibitor of formula (Formula-1)

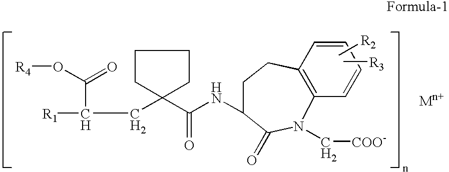

wherein:
R₁ is a (C₁-C₆) alkoxy(C₁-C₆) alkyl group, which is optionally substituted by a group chosen from a (C₁-C₆) alkoxy group, a phenyl-(C₁-C₆)-alkyl group and a phenyloxy-(C₁-C₆)-alkyl group, wherein the phenyl group is optionally substituted with a substituent chosen from a (C₁-C₆)alkyl group, a (C₁-C₆) alkoxy group, a halogen atom, and a naphtyl-(C₁-C₆)-alkyl group,
R₂ and R₃, which are the same or different, are chosen from a hydrogen atom and a halogen atom,
R₄ is a biolabile ester forming group,
M is chosen from a hydrogen atom and a metal ion, such as a bivalent metal ion, and
n is chosen from 1, 2 and 3;

In another embodiment of the present invention, the pharmaceutical composition comprises an alkali system comprising a mixture of at least two alkaline compounds in a ratio of from 1:20 to 20:1 and at least one active agent, wherein the active agent is chosen from an endothelin conversion enzyme (ECE) inhibitor and a neutral endopeptidase (NEP) inhibitor, SLV-306, of chemical formula 3-(1-(2′-(Ethoxycarbonyl)-4′-phenyl-butyl)-cyclopentan-1-carbonylamino)-2,3,4,5-tetrahydro-2-oxo-1H-1-benzazepin-1-acetic acid, or at least one pharmaceutically acceptable salt, ester, hydrate, solvate, isomer or derivative thereof.

In another embodiment of the present invention, the pharmaceutical composition comprises an alkali system comprising a mixture of at least two alkaline compounds in a ratio of from 1:20 to 20:1 w/w and an active agent, wherein the active agent is SLV-306 in its calcium salt form.

In yet another embodiment of the present invention, the pharmaceutical composition comprises an alkali system comprising a mixture of at least two alkaline compounds in a ratio of from 1:20 to 20:1 w/w, and an active agent, wherein the active agent is SLV-306 calcium salt in its 3S,2′R form. This compound is referred to as Compound S—Ca, the corresponding acid (1H-1-Benzazepine-1-acetic acid, 3-[[[1-[2-(ethoxycarbonyl)-4-phenylbutyl]-cyclopentyl]carbonyl]amino]-2,3,4,5-tetrahydro-2-oxo-) is referred to as Compound S—H, and the corresponding sodium salt is referred to as Compound S—Na.

In one embodiment, the active agent of Formula-1 is present in the composition in an amount from about 10% to 80% by weight of the composition, such as from about 15 to 75% by weight of the composition. The active agent is or may optionally be used in a micronized form.

In another embodiment, the alkali system comprises a mixture of sodium bicarbonate and sodium carbonate (Effer-Soda™-12) marketed by SPI Pharma. Effer-Soda™-12 is a highly stable, surface modified sodium bicarbonate powder; it is produced by converting the surface of sodium bicarbonate particles to sodium carbonate. Primarily, Effer-Soda™-12 contains 83-90% w/w sodium bicarbonate and 10-17% w/w sodium carbonate. The outer layer of sodium carbonate absorbs moisture (from the atmosphere or composition) and forms sodium sesquicarbonate, which is stable up to 70° C. temperature. This protection mechanism provided by the heat stable sodium sesquicarbonate prevents early effervescent reaction at ambient and elevated temperature storage conditions.

Surprisingly the inventors of the present invention have found that using an alkaline compound in the formulation, alone or in a mixture, e.g., Effer-Soda™-12, even without any surfactant in the composition prevents the difficult to solubilize gel formation in the acid gastric fluid, thereby enhancing the solubility of SLV-306, as evidenced during in vitro dissolution studies in a biphasic dissolution model (see Example 1a), which indicates an improvement in the in vivo solubility as well and thus improvement in bioavailability. Further, the compositions have a good stability upon storage. Further, since the Effer-Soda™-12 is granular in nature, its use in formulating the compositions of the present invention has improved the flow properties and compressibility of material used to formulate the desired dosage form and also improved its machinability.

Specific solid alkaline compounds like the bicarbonates and carbonates as indicated above may be used in combination with solid acidic compounds (e.g., citric acid, tartaric acid, adipic acid, fumaric acid, succinic acid, ascorbic acid, nicotinic acid, saccharin, aspirin, malic acid, sodium dihydrogen phosphate, disodium dihydrogen pyrophosphate, sodium dihydrogen citrate and disodium hydrogen citrate) in effervescent compositions. In some embodiments of the present invention, the composition does not contain an acidic compound.

In another embodiment of the present invention, the pharmaceutical compositions of present invention optionally comprise at least one pharmaceutically acceptable excipient chosen from, but not limited to, diluents, disintegrants, binders, polymers, solubilizers, fillers, bulking agents, anti-adherants, anti-oxidants, buffering agents, colorants, flavoring agents, coating agents, plasticizers, surfactants, organic solvents, stabilizers, preservatives, lubricants, glidants, chelating agents, and the like known to the art used either alone or in combination thereof.

Diluents that can be used in the present invention include lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, and the like or mixtures thereof.

Binders that can be used in the present invention include acacia, alginic acid and salts thereof, cellulose derivatives, methylcellulose, hydroxyethyl cellulose, hydroxypropyl cellulose, polyethylene glycol, gums, polysaccharide acids, gelatin, polyvinylpyrrolidone, polyvinylpyrrolidone/vinyl acetate copolymer, polymethacrylates, hydroxypropyl-methylcellulose, ethylcellulose, starch, pregelatinized starch, tragacanth, dextrin, microcrystalline cellulose, sucrose, or glucose, and the like or mixtures thereof.

Disintegrants useful in the present invention are selected from, but not limited to, starches, pregelatinized starch, celluloses, cross-linked carboxymethylcellulose, crospovidone, crosslinked polyvinylpyrrolidone, a calcium or a sodium alginate complex, clays, alginates, sodium starch glycolate, croscarmellose sodium and the like or mixtures thereof.

Lubricants that can be used in the present invention include magnesium stearate, sodium stearyl fumarate, hydrogenated vegetable oil, stearic acid, glyceryl behenate, stearates, waxes and the like or mixtures thereof can be used. Stabilizers such as antioxidants, buffers, or acids, and the like are useful in the present invention. Glidants such as talc, colloidal silicon dioxide or the like.

Polymers that can also be used in formulating a composition of the present invention include cellulosic derivatives, polyalkylene oxides, acrylic acid and methacrylic acid polymers, crosslinked polyacrylic acids, polysaccharide gums such as xanthan gum, veegum, agar, guar gum, locust bean gum, gum arabic, okra gum, alginic acid, alginates, bentonite, arabinoglactin, pectin, tragacanth, scleroglucan, dextran, amylose, amylopectin, dextrin, and the like or mixtures thereof. Solubilizers that can be used in the present invention include polyethylene glycol and their derivatives, for example, Gelucire® such as Gelucire® 50/13 (Gattefosse); polyoxyethylene alkyl ethers such as polyoxyethylene stearyl ether, polyoxyethylene oleyl ether and polyoxyethylene cetyl ether which are available under the Brij® and Cetomacrogol® series trade names; polyvinylpyrrolidone K-30, polyvinylpyrrolidone K-90 or Kollidon® VA 64; polar solvent; and the like used either alone or in combination.

The present invention also relates to a process for preparing the formulation as described above. In a first embodiment of this aspect of the present invention, the process for the preparation of such improved compositions comprises:
i) mixing the active agent and alkali system optionally with at least one pharmaceutically acceptable excipient, thereby forming a mixture; and
ii) formulating the mixture produced in (i) into a suitable dosage form.

In another embodiment of this process of the present invention, the process comprises:
i) mixing the active agent, alkali system, and a lubricant,
ii) optionally adding at least one pharmaceutically acceptable excipient, thereby forming a mixture; and
iii) formulating the mixture produced in (i) and (ii) into a suitable dosage form.

In a further embodiment of the process of the present invention, the process comprises:
i) mixing SLV-306 or at least one pharmaceutically acceptable salt, ester, hydrate, solvate, isomer or derivative; the alkali system, the disintegrant and the lubricant,
ii) optionally adding at least one additional pharmaceutically acceptable excipient, forming a mixture, and
iii) formulating the mixture produced in (i) and (ii) into a suitable dosage form.

In a further embodiment, the composition of the present invention is in the form of a solid dosage form, such as tablets, capsules, patches or the like. The tablets can be prepared by either direct compression, dry compression (slugging), or by granulation. In one embodiment of the present invention, the oral composition is prepared by compression or compaction. The granulation technique is either aqueous or non-aqueous. The non-aqueous solvent used is selected from ethanol, isopropyl alcohol, ethyl acetate, methyl t-butyl ether (MTBE), and methylene chloride. In one embodiment, the composition of the present invention are in the form of compacted tablets, compressed tablets, molded tablets, and the like.

When the formulations of the present invention are provided in the form of tablets, these tablets have disintegration times of from 5 minutes to 90 minutes, for example, below 60 minutes and below 45 minutes. Formulations with short disintegration times can be prepared by using a mixture of sodium bicarbonate and sodium carbonate as available, e.g., in Effer-Soda™-12.

The present invention also provides a method for treating at least one disease chosen from chronic heart failure, myocardial infarction, cardiogenic shock, systemic and pulmonary hypertension, ischemia-repurfusion injury, atherosclerosis, coronary and systemic vasospastic disorders, cerebral vasospasm, and subarachnoid hemorrhage, the method comprising administering an effective amount of the inventive composition to a patient in need thereof.

The improved compositions of the present invention are easier to formulate and possess improved solubility and stability.

The following examples are only intended to further illustrate the invention, in more detail, and therefore these Examples are not deemed to restrict the scope of the invention in any way.

### EXAMPLES

### Example 1

### Materials and Methods

Materials

S—Ca was prepared according to the prescription given in Examples 2 and 3 of WO03/059939 starting with the acid prepared according to Example 2 of EP 0733642. In all the Examples the actual amount of S—Ca is given. 103.75 mg S—Ca corresponds with 100 mg S—H which is the active principle. Sodium bicarbonate was obtained from Sigma Aldrich or Canton Labs, India. Effer-Soda™-12 was obtained from SPI Pharma, Newcastle, Del. US. All other auxiliary materials that were used are readily commercially available.

Methods

Description of the Bi-Phase In-Vitro Dissolution Method.

The bi-phase dissolution was performed with the USP apparatus 2 configuration. The paddle speed was 50 rpm and the temperature of the vessels (and so the dissolution medium) was maintained at 37.0° C. using Vankel VK7010 equipment. The dissolution of the formulations was started in 500 ml 0.1 M hydrochloric acid (4.2 ml concentrated hydrochloric acid (HCl) in 500 ml water)(phase 1). After 0, 5, 15 and 30 minutes a sample was taken. After 30 minutes 500 ml 1 M phosphate buffer (32.4 gram sodium di-hydrogen phosphate NaH₂PO₄ and 124.8 gram di-sodium hydrogen phosphate (Na₂HPO₄) in 1000 ml water was added to phase 1. Addition of the phosphate buffer changed the pH of the dissolution medium from pH 1 in phase 1 to pH 6.8 in phase 2. During the dissolution test the pH of both phases remained unchanged. Samples were taken after 35, 45 and 60 minutes. All the samples were filtered through a Pall Zymark Acrodisc PSF, GxF/GHP, 0.45 μm or a Millipore Millex-FH (hydrophobic PTFE 0.45 μm) filter. The quantity of the dissolved daglutril in the filtered samples was analyzed by off-line UV measurements at 240 nm using external standardization. In an earlier comparative study with the calcium salt of the compound SLV306 (S—Ca), it was shown that this bi-phase in vitro dissolution method had a good correlation with in-vivo results.

### Example 2

### Preparation of a Traditionally Formulated Coated Tablet of SLV-306

| Ingredients | Quantity (mg/tablet) |
|---|---|
| S—Ca | 414.25 |
| Micro crystalline cellulose PH301 | 249.00 |
| Cross-linked polyvinylpyrrolidon | 14.00 |
| Sodium stearyl fumarate | 1.75 |
| Opadry II Yellow coating | 21.00 |
| Tablet weight | 700.00 |

Procedure
i) S—Ca was compacted and the compact was passed through a 1.0 mm sieve.
ii) The material of step (i) was mixed with micro crystalline cellulose PH301, cross-linked polyvinylpyrrolidone and sodium stearyl fumarate to obtain a uniform mixture.
iii) The material of step (ii) was compressed using a tablet compression machine.
iv) The tablets from step (iii) were coated in suitable coating equipment.

### Example 3

### Preparation of Tablets of SLV-306 Containing Effer-Soda™-12

Quantity (mg/tablet)

| Ingredients | Tablet I | Tablet II |
|---|---|---|
| S—Ca | 622.5 | 622.5 |
| Effer-Soda ™-12 | 299.5 | 599.5 |
| Magnesium stearate | 10.0 | 13.0 |
| Sodium starch glycolate | 33.0 | 65.0 |
| Opadry II Yellow coating | 35.0 | 47.2 |
| Tablet weight | 1000.0 | 1347.2 |

Procedure
i) The S—Ca, Effer-Soda™-12, Magnesium stearate and Sodium starch glycolate were sifted through an appropriate sieve, e.g. a #40 mesh sieve.
ii) The S—Ca, Effer-Soda™-12 and a portion of Magnesium stearate and Sodium starch glycolate sifted above were mixed to obtain a uniform mixture.
iii) The material of step (ii) was compacted and the compact was passed through an appropriate sieve, e.g. a #30 mesh sieve.
iv) The material of step (iii) was mixed with the remaining quantity of Magnesium stearate and Sodium starch glycolate.
v) The material of step (iv) was compressed using a tablet compression machine
vi) The tablets of step (v) were coated by spraying an Opadry II Yellow 85F22185 aqueous suspension on the tablets, followed by drying of the tablets.

### Example 4

### Comparative Dissolution Study for SLV306 Formulation with Effer-Soda™-12 and a Traditionally Formulated Tablet

A comparative dissolution study according to the method described in Example 1 was carried out on one batch of a traditionally formulated tablet (Tablet A, prepared as described in Example 2) and two batches of the calcium salt of SLV-306 (S—Ca) (Tablet B, prepared as described in Example 3 (I) and Tablet C, prepared as described in Example 3 (II)).

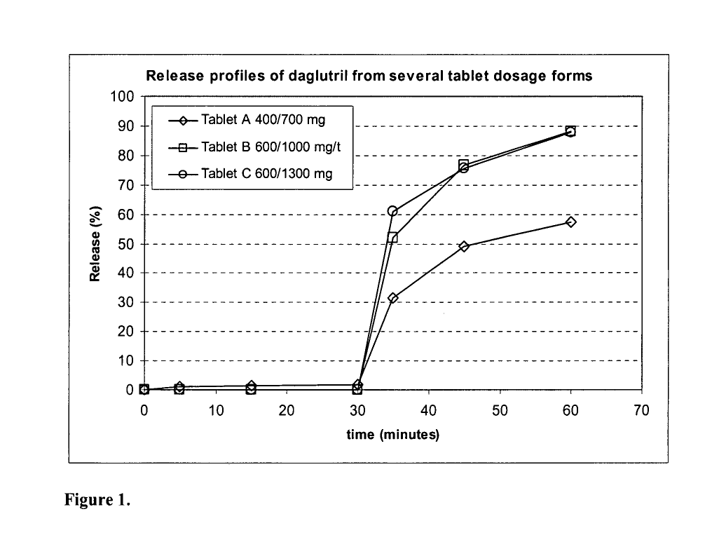

The release profile of these formulations is given in the Table below and depicted in FIG. 1. From this study it was concluded that a formulation of S—Ca with a high drug load and a favorable release profile can be prepared.

| Time | Drug Release in % | | |
|---|---|---|---|
| (min) | Tablet A | Tablet B | Tablet C |
| 0 | 0 | −0.02 | 0.00 |
| 5 | 1.1 | −0.02 | 0.10 |
| 15 | 1.6 | −0.03 | 0.15 |
| 30 | 1.9 | −0.07 | 0.08 |
| 35 | 31.4 | 51.99 | 60.88 |
| 45 | 49.1 | 76.76 | 75.73 |
| 60 | 57.4 | 88.27 | 87.79 |

### Example 5

### Preparation of Film-Coated Tablets of SLV-306 Containing Effer-Soda

| Ingredients | Quantity (mg/tablet) |
|---|---|
| S—Ca | 311.25 |
| Effer-Soda ™-12 | 300.00 |
| Microcrystalline cellulose (Avicel ® | 310.00 |
| Croscarmellose sodium | 20.00 |
| Isopropyl alcohol | q.s (lost in processing) |
| Hydrogenated castor oil | 7.50 |
| Purified talc | 7.50 |
| Colloidal silicon dioxide | 7.50 |
| Opadry II Yellow 85F22185 | 30.00 |
| Purified water | q.s. (lost in processing) |

Procedure
i) S—Ca, Effer-Soda™-12, Microcrystalline cellulose (Avicel® PH 101) and Croscarmellose sodium were sifted through an appropriate sieve, e.g., a #40 mesh sieve and mix.
ii) The mixture was granulated using isopropyl alcohol followed by sifting through an appropriate sieve, e.g., a #24 mesh sieve and drying.
iii) Hydrogenated castor oil (Lubritab®), Purified talc and Colloidal silicon dioxide were sifted through an appropriate sieve, e.g. a #40 mesh sieve and mixed.
iv) The material of step (iii) was added to the material of step (ii) and mixed.
v) The material of step (iv) was compressed, using a tablet compression machine.
vi) The tablets of step (v) were coated by spraying an Opadry II Yellow 85F22185 suspension in water on the tablets followed by drying of the tablets.

### Example 6

### Preparation of Capsules of SLV-306

| Ingredients | Quantity (mg/capsule) |
|---|---|
| S—Ca | 311.25 |
| Magnesium carbonate | 150.00 |
| Dicalcium phosphate | 131.25 |
| Sodium starch | 30.00 |
| Magnesium stearate | 10.00 |

Procedure
i) S—Ca, Magnesium carbonate, Dicalcium phosphate, Sodium starch glycolate and Magnesium stearate were sifted through an appropriate sieve, e.g., a #40 mesh sieve, and mixed.
ii) The material of step (i) was compacted and the compacts were passed through a #30 mesh sieve.
iii) The material of step (ii) was lubricated with #60 mesh sieve passed Magnesium stearate.
iv) The material of step (iii) was filled into a hard gelatin capsule.

What is claimed is:

1. An oral pharmaceutical composition comprising an active agent in an amount greater than 10% w/w of the composition, wherein the active agent comprises at least one endothelin conversion enzyme (ECE) inhibitor or neutral endopeptidase (NEP) inhibitor, an alkali system in an amount greater than 10% w/w of the composition, and optionally at least one pharmaceutically acceptable excipient, with the proviso that the active agent is not a compound of formula (I): 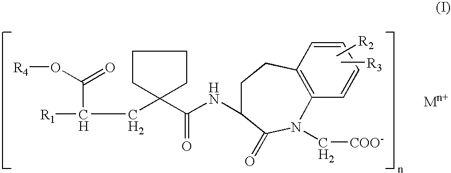
or a pharmaceutically acceptable salt, ester, hydrate, solvate, isomer or derivative thereof,
wherein:
R₁ is a (C₁-C₆) alkoxy(C₁-C₆) alkyl group which is optionally substituted with at least one of a (C₁-C₆) alkoxy group, a phenyl-(C₁-C₆)-alkyl group or a phenyloxy-(C₁-C₆)-alkyl group, wherein the phenyl group is optionally substituted with at least one of a (C₁-C₆)alkyl group, a (C₁-C₆) alkoxy group, a halogen atom, or a naphtyl-(C₁-C₆)-alkyl group;
R₂ and R₃, which are the same or different, are chosen from a hydrogen atom and a halogen atom;
R₄ is a biolabile ester forming group;
M is chosen from a hydrogen atom and a metal ion; and
n is chosen from 1, 2 and 3.

2. The composition according to claim 1, wherein M is a bivalent metal ion.

3. The composition according to claim 1, wherein the active agent is chosen from CGS 26303, phosphoramidon, FR901533, TMC-66, SM-19712, KC-12615, KC-90095-1-AC, CGS-26303, CGS-30440, CGS-31447, CGS-26670, and Sch-54470, and the pharmaceutically acceptable salts, esters, isomers, derivatives and prodrugs thereof.

4. The composition according to claim 1, wherein the alkali system is chosen from sodium bicarbonate, sodium carbonate, potassium bicarbonate, potassium carbonate, magnesium carbonate, calcium carbonate, tris buffer, triethanolamine; an alkaline hydroxide, an alkaline phosphate, and meglumine and mixtures thereof.

5. The composition according to claim 4, wherein the alkaline hydroxide is chosen from sodium hydroxide, potassium hydroxide and magnesium hydroxide.

6. The composition according to claim 4, wherein the alkaline phosphate is chosen from disodium hydrogen phosphate, dipotassium hydrogen phosphate and dicalcium phosphate.

7. The composition according to claim 1, wherein the alkali system comprises a mixture of at least two alkaline compounds.

8. The composition according to claim 7, wherein the alkali system comprises a mixture of sodium bicarbonate and sodium carbonate.

9. An oral pharmaceutical composition comprising an active agent in an amount greater than 10% w/w of the composition, wherein the active agent comprises at least one endothelin conversion enzyme (ECE) inhibitor or neutral endopeptidase (NEP) inhibitor, an alkali system comprising a mixture of at least two alkaline compounds in a ratio of from 1:20 to 20:1 w/w, wherein the alkali system comprises an amount greater than 10% w/w of the composition, and optionally at least one pharmaceutically acceptable excipient.

10. The composition according to claim 9, wherein the active agent is chosen from CGS 26303, phosphoramidon, FR901533, TMC-66, SM-19712, KC-12615, KC-90095-1-AC, CGS-26303, CGS-30440, CGS-31447, CGS-26670, and Sch-54470, and the pharmaceutically acceptable salts, esters, isomers, derivatives and prodrugs thereof.

11. The composition according to claim 9, wherein the active agent comprising at least one endothelin conversion enzyme (ECE) inhibitor or neutral endopeptidase (NEP) inhibitor is an active agent of formula (I) 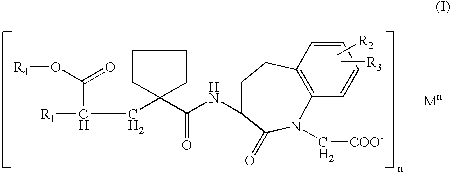
or at least one pharmaceutically acceptable salt, ester, hydrate, solvate, isomer or derivative thereof,
wherein:
R₁ is a (C₁-C₆) alkoxy(C₁-C₆) alkyl group, which is optionally substituted with at least one of a (C₁-C₆) alkoxy group, a phenyl-(C₁-C₆)-alkyl group or a phenyloxy-(C₁-C₆)-alkyl group, wherein the phenyl group is optionally substituted with at least one of a (C₁-C₆)alkyl group, a (C₁-C₆) alkoxy group, a halogen atom, or a naphtyl-(C₁-C₆)-alkyl group;
R₂ and R₃, which are the same or different, are chosen from a hydrogen atom and a halogen atom;
R₄ is a biolabile ester forming group;
M is chosen from a hydrogen atom and a metal ion; and
n is chosen from 1, 2 and 3.

12. The composition as claimed in claim 11, wherein M is a bivalent metal ion.

13. The composition according to claim 11, wherein M is calcium in its 2+ form.

14. The composition according to claim 11, wherein the active agent is the calcium salt of 1H-1-Benzazepine-1-acetic acid 3-[[[1-[2-(ethoxycarbonyl)-4-phenylbutyl]-cyclopentyl]carbonyl]-amino]-2,3,4,5-tetrahydro-2-oxo-.

15. The composition according to claim 14, wherein the calcium salt of 1H-1-Benzazepine-1-acetic acid 3-[[[1-[2-(ethoxycarbonyl)-4-phenylbutyl]-cyclopentyl]carbonyl]-amino]-2,3,4,5-tetrahydro-2-oxo- is in its 3S,2′R form.

16. The composition according to claim 11, wherein the alkali system comprises a mixture of two alkaline compounds.

17. The composition according to claim 16, wherein the alkali system comprises a mixture of sodium bicarbonate and sodium carbonate.

18. The composition according to claim 17, wherein the alkali system comprises from 83 to 90% w/w of sodium bicarbonate and from 10 to 17% w/w of sodium carbonate.

19. The composition according to claim 1, wherein the alkali system is present in an amount of at least 20% w/w of the composition.

20. The composition according to claim 11, wherein the alkali system is present in an amount of at least 20% w/w of the composition.

21. The composition according to claim 1, wherein the at least one pharmaceutically acceptable excipient is chosen from diluents, disintegrants, binders, polymers, solubilizers, fillers, bulking agents, anti-adherants, anti-oxidants, buffering agents, colorants, flavoring agents, coating agents, plasticizers, organic solvents, stabilizers, preservatives, lubricants, glidants, and chelating agents, and combinations thereof.

22. The composition according to claim 11, wherein the at least one pharmaceutically acceptable excipient is chosen from diluents, disintegrants, binders, polymers, solubilizers, fillers, bulking agents, anti-adherants, anti-oxidants, buffering agents, colorants, flavoring agents, coating agents, plasticizers, organic solvents, stabilizers, preservatives, lubricants, glidants, and chelating agents, and combinations thereof.

23. The composition according to claim 1, wherein the composition is in the form of granules, tablets or capsules.

24. The composition according to claim 11, wherein the composition is in the form of granules, tablets or capsules.

25. A process for preparation of a composition according to claim 1, said process comprising:
i) mixing an active agent and an alkali system optionally with at least one pharmaceutically acceptable excipient, and
ii) formulating the mixture produced in (i) into a suitable dosage form.

26. A process for preparation of a composition according to claim 1, said process comprising:
i) mixing an active agent, an alkali system, and a lubricant,
ii) optionally adding at least one pharmaceutically acceptable excipient, and
iii) formulating the mixture produced in (i) and (ii) into a suitable dosage form.

27. A process for preparation of a composition according to claim 11, said process comprising:
i) mixing an active agent and an alkali system optionally with at least one pharmaceutically acceptable excipient, and
ii) formulating the mixture produced in (i) into a suitable dosage form.

28. A process for preparation of a composition according to claim 11, said process comprising:
i) mixing an active agent, an alkali system, and a lubricant,
ii) optionally adding at least one other pharmaceutically acceptable excipient, and
iii) formulating the mixture produced in (i) and (ii) into a suitable dosage form.

29. A process for preparation of a composition according to claim 11, said process comprising:
i) mixing SLV-306 or at least one pharmaceutically acceptable salt, ester, hydrate, solvate, isomer or derivative, an alkali system, a disintegrant and a lubricant,
ii) optionally adding at least one pharmaceutically acceptable excipient, and
iii) formulating the mixture produced in (i) and (ii) into a suitable dosage form.

30. A method for treating at least one disease chosen from chronic heart failure, myocardial infarction, cardiogenic shock, systemic and pulmonary hypertension, ischemia-repurfusion injury, atherosclerosis, coronary and systemic vasospastic disorders, cerebral vasospasm, and subarachnoid hemorrhage, the method comprising administering an effective amount of a composition according to claim 11 to a patient in need thereof.

31. A method for treating at least one disease chosen from chronic heart failure, myocardial infarction, cardiogenic shock, systemic and pulmonary hypertension, ischemia-repurfusion injury, atherosclerosis, coronary and systemic vasospastic disorders, cerebral vasospasm, and subarachnoid hemorrhage, the method comprising administering an effective amount of a composition according to claim 1 to a patient in need thereof.
